(19) United States
(12) Patent Application Publication
(10) Pub. No.: US 2013/0164249 A1
(43) Pub. Date: Jun. 27, 2013

(54) COMPOSITIONS AND METHODS FOR INHIBITING VIRAL AND BACTERIAL ACTIVITY

(71) Applicants: Patrick F. Kiser, Salt Lake city, UT (US); Julie I. Jay, Salt Lake City, UT (US); Meredith C. Roberts Clarck, Salt Lake City, UT (US)

(72) Inventors: Patrick F. Kiser, Salt Lake city, UT (US); Julie I. Jay, Salt Lake City, UT (US); Meredith C. Roberts Clarck, Salt Lake City, UT (US)

(21) Appl. No.: 13/774,805

(22) Filed: Feb. 22, 2013

Related U.S. Application Data
(63) Continuation of application No. 12/666,432, filed on Nov. 22, 2010, now abandoned.
(60) Provisional application No. 60/946,640, filed on Jun. 27, 2007.

(51) Int. Cl. A61K 31/80; A61K 45/00

(52) U.S. Cl. CPC A61K 31/80; A61K 45/00
USPC 424/78.35; 435/238; 435/252.1

(57) ABSTRACT

Described herein are methods of inhibiting the activity of a virus or a bacterium comprising contacting a virus or bacterium with a polymer functionalized with at least one boronic acid moiety. Also described are methods of treatment using such functionalized polymers.

### CROSS-REFERENCE TO RELATED PATENT APPLICATIONS

This application is a continuation of U.S. patent application Ser. No. 12/666432, filed Nov. 22, 2010, which claims the benefit of priority as a national stage application of PCT/US2008/068306, filed Jun. 26, 2008, which claims priority to U.S. Provisional Application 60/946640, filed Jun. 27, 2007, the entire contents of all of which are incorporated herein by reference in their entireties for any and all purposes.

### FIELD OF THE INVENTION

The present invention relates to compositions and methods for inhibiting viral and bacterial activity, particularly viral and bacterial infections. More specifically, the present invention relates to the use of polymers functionalized with boronic acid groups to inhibit the activity of viruses and bacteria and/or treat viral and bacterial infections.

### SUMMARY OF THE INVENTION

Compositions and methods have been discovered for inhibiting the activity of infectious organisms such as viruses and bacteria. In one aspect, the invention provides methods for inhibiting the activity of a virus or bacterium which include contacting the virus or bacterium with a polymer functionalized with boronic acid groups whereby the activity of the virus or bacterium is inhibited. In a further aspect, there are provided methods for treating an infection, such as a viral or bacterial infection comprising administering to a subject suffering from an infection, a composition comprising a polymer functionalized with boronic acid moieties.

In some embodiments of inventive methods, the one or more boronic acid moieties comprises an alkylboronic acid group. In other embodiments, the one or more boronic acid moieties comprises an arylboronic acid such as a substituted or unsubstituted phenylboronic acid. The aryl boronic acid groups may be optionally substituted with one or more groups, e.g., electron withdrawing groups such as halogen, cyano, nitro, hydroxymethyl, aminomethyl and the like. In some embodiments, the substituents are ortho hydroxymethyl groups. In other embodiments, the substitutents are ortho aminomethyl groups. In still other embodiments, the one or more boronic acid moieties comprises a spacer moiety. The spacer moiety can be selected from the group consisting of alkyl, polyether, ester, diester, amide and diamide groups. For example, the spacer moiety can be —C(O)NH(CH₂)ₚ₋, where p is from 1 to 10 or it can be —C(O)NH(CH₂)ₚNHC(O)—, where p is from 1 to 10.

Many different polymers may be used in methods disclosed herein including homopolymers, copolymers and multi-armed polymers. The polymer can comprise one or more polymers selected from polyethylene oxide and polypropylene oxide, or from polyethylene, polypropylene, polybutylene, polystyrene, polyamide, polyacrylonitrile, polyester and polyurethane. Other suitable polymers contemplated for use in the present methods include one or more polymers selected from the polyacrylate, poly(meth)acrylate, poly(hydroxymethyl methacrylate), poly(hydroxyethyl methacrylate), poly(hydroxypropyl methacrylate), poly(alkylcyanoacrylate), polyethylene glycol diacrylate, polyethylene glycol dimethacrylate, polyacrylamide, poly(N-isopropyl acrylamide, poly(meth)acrylamide and poly(hydroxypropyl methacrylamide). In some embodiments, the polymer comprises 2-hydroxypropyl methacrylamide, and in others the polymer comprises 2-hydroxypropyl methacrylamide and the one or more boronic acid groups is phenylboronic acid. In some embodiments, the 2-hydroxypropyl methacrylamide is present in a percent molar ratio to phenylboronic acid of from about 85:15 to about 99:1. In other embodiments, the ratio is from about 5:95 to about 95:5.

In some methods of inhibiting the activity of a virus or bacterium, the virus or bacterium is capable of causing a sexually transmitted infection. For example, in some methods, the virus is HIV. In certain embodiments the polymer covalently binds to the virus. The inhibited activity can be entry of the virus into a cell in some embodiments.

In some methods of treating an infection, the infection may be a viral infection or a bacterial infection. In some embodiments, the infection being treated is a sexually transmitted infection and may be bacterial or viral, such as an HIV infection. Methods of treating infection may further include administering a bioactive agent, wherein the bioactive agent is selected from the group consisting of anti-inflammatory agents, antibacterial agents, analgesic agents, local anesthetics, immunogens, hormones, contraceptive agents, antivirals, antifungals and antihistamines. The composition can be administered topically to the subject and may be in the form of an ointment, lotion, cream, gel, foam, gelcap, drop or suppository. In some embodiments, the composition is administered to the vagina, penis or rectum of the subject. For example, the composition may be administered using an intravaginal sponge or ring, or may be applied to a condom.

Further features and advantages of the present compositions and methods will be apparent from the following detailed description and drawings.

### BRIEF DESCRIPTION OF THE DRAWINGS

FIG. 1 is a schematic illustrating the inhibition of viral activity by a polymer functionalized with boronic acid moieties. Upon contacting the functionalized polymer with the virus, the boronic acid moieties covalently bind with the sugar residues present on the surface of the virus, thereby inactivating the virus.

FIG. 2 shows the structure of a polymer containing 90 mol % 2-hydroxypropylmethacrylamide (HPMA) and 10 mol % phenylboronic acid (PBA).

FIG. 3 shows chemical structures of the small molecule boronic acid ligands and affinity results for a HIV-1 CN54 gp120 functionalized surface from surface plasmon resonance: a) 2,5-difluorophenylboronic acid (1) was used as an example of a PBA with a low pKa due to electron withdrawing substituents on the aromatic ring while ortho-hydroxymethyl phenylboronic acid (2) was utilized due to the ortho-hydroxymethyl substituent. b) Raw SPR data for 1 at pH 7.5 comparing response of the ligand binding to the gp120 functionalized surface (right) and no binding to the nonfunctionalized dextran surface (left). c) Apparent affinity, K_{D}, at 25° C. of 1 and 2 to gp120 as a function of pH and in the presence of fructose. The presence of the ortho-hydroxymethyl is thought to facilitate formation of the dehydrated boronophthalide form as shown above. d) Binding curves of PBA ligands produced from SPR analysis based on a two fold dilution series. 2 was not soluble at pH 5.5. N=2, mean±SD.

FIG. 4 shows results from neutralization assay of polymer solutions 5a and 5b in buffer (Left) compared to buffer alone (Right) under serial dilutions with growth media.

FIG. 5 shows results from cytotoxicity assay of polymer solutions 5a and 5b in buffer (Left) compared to buffer alone (Right) under serial dilutions with growth media.

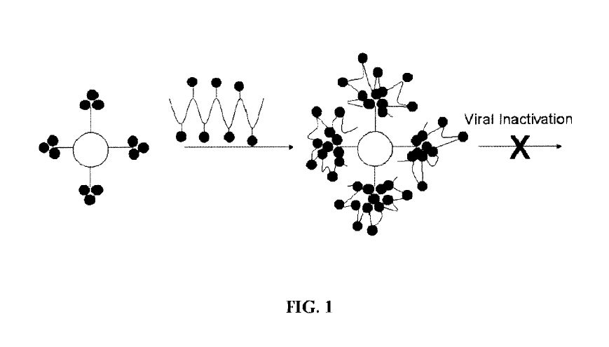

### DETAILED DESCRIPTION OF THE PREFERRED EMBODIMENTS

In this specification and in the claims that follow, reference will be made to a number of terms that shall be defined to have the following meanings:

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the polymer” includes mixtures of two or more such polymers, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed, then “less than or equal to” the value, “greater than or equal to the value,” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application data are provided in a number of different formats and that these data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

References in the specification and concluding claims to parts by weight of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound

A weight percent of a component, unless specifically stated to the contrary, is based on the total weight of the formulation or composition in which the component is included.

As used herein, the term “substituted” is contemplated to include all permissible substituents of organic compounds. In a broad aspect, the permissible substituents include functional groups and acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. In some embodiments the alkyl group has 1 to 18, 1 to 12, or 1 to 8 carbons. A “lower alkyl” group is an alkyl group containing from one to six carbon atoms. The alkyl group can also be substituted or unsubstituted. The alkyl group can be substituted with one or more groups including, but not limited to, substituted or unsubstituted alkyl, cycloalkyl, alkoxy, alkenyl, cycloalkenyl, alkynyl, cycloalkynyl, heterocyclyl, aryl, heteroaryl, aldehyde, amide, amino, carboxylic acid, boronic acid, ester, ether, halide, hydroxy, ketone, azide, nitro, hydroxamate, silyl, sulfo-oxo, or thiol, as described herein.

The term “cycloalkyl” as used herein is a non-aromatic carbon-based cyclic group composed of at least three carbon atoms. In some embodiments, the cycloalkyl group has from 3 to 14 carbons, 3 to 10, or 3 to 8 carbons. Cycloalkyl groups include monocyclic, bicyclic and tricyclic rings. Cycloalkyl groups having two or three rings include fused and bridged ring systems. Examples of cycloalkyl groups include, but are not limited to, cyclopropyl, cyclobutyl, cyclopentyl, cyclohexyl, norbornyl, and the like. Cycloalkyl groups can be unsubstituted or substituted with one or more groups including, but not limited to, substituted or unsubstituted alkyl, cycloalkyl, alkoxy, alkenyl, cycloalkenyl, alkynyl, cycloalkynyl, heterocyclyl, aryl, heteroaryl, aldehyde, amide, amino, carboxylic acid, boronic acid, ester, ether, halide, hydroxy, ketone, azide, nitro, hydroxamate, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The term “alkoxy” as used herein is an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. In some embodiments, alkenyl groups include 2 to 18, 2 to 12, 2 to 8, or 2 to 6 carbons. Asymmetric structures such as (A¹A²)C═C(A³A⁴) are intended to include both the E and Z isomers. This may be presumed in structural formulae herein wherein an asymmetric alkene is present, or it may be explicitly indicated by the bond symbol C═C. Examples of alkenyl groups include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, and the like. The alkenyl group can be substituted with one or more groups including, but not limited to, substituted or unsubstituted alkyl, cycloalkyl, alkoxy, alkenyl, cycloalkenyl, alkynyl, cycloalkynyl, heterocyclyl, aryl, heteroaryl, aldehyde, amide, amino, carboxylic acid, boronic acid, ester, ether, halide, hydroxy, ketone, azide, nitro, hydroxamate, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The cycloalkenyl group can be unsubstituted or substituted with one or more groups including, but not limited to, substituted or unsubstituted alkyl, cycloalkyl, alkoxy, alkenyl, cycloalkenyl, alkynyl, cycloalkynyl, heterocyclyl, aryl, heteroaryl, aldehyde, amide, amino, carboxylic acid, boronic acid, ester, ether, halide, hydroxy, ketone, azide, nitro, hydroxamate, silyl, sulfo-oxo, or thiol as described herein.

The term “alkynyl” as used herein is a hydrocarbon group of 2 to 24 carbon atoms with a structural formula containing at least one carbon-carbon triple bond. In some embodiments, the alkynyl group has 2 to 18, 2 to 12, 2 to 8 or 2 to 6 carbons. Exemplary alkynyl groups include but are not limited to —C≡CH, —C≡CCH₃, —CH₂C≡CH, and the like. The alkynyl group can be unsubstituted or substituted with one or more groups including, but not limited to, substituted or unsubstituted alkyl, cycloalkyl, alkoxy, alkenyl, cycloalkenyl, alkynyl, cycloalkynyl, heterocyclyl, aryl, heteroaryl, aldehyde, amide, amino, carboxylic acid, boronic acid, ester, ether, halide, hydroxy, ketone, azide, nitro, hydroxamate, silyl, sulfo-oxo, or thiol, as described herein.

The term “cycloalkynyl” as used herein is a non-aromatic carbon-based ring composed of at least seven carbon atoms and containing at least one carbon-carbon triple bound. In some embodiments, the cycloalkynyl group has from 7 to 12 carbons. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, substituted or unsubstituted alkyl, cycloalkyl, alkoxy, alkenyl, cycloalkenyl, alkynyl, cycloalkynyl, heterocyclyl, aryl, heteroaryl, aldehyde, amide, amino, carboxylic acid, boronic acid, ester, ether, halide, hydroxy, ketone, azide, nitro, hydroxamate, silyl, sulfo-oxo, or thiol as described herein.

The term “aryl” as used herein is a group that contains any carbon-based aromatic group having from 6 to 14 carbons. Aryl groups include bicyclic and tricyclic ring systems that may be fused or not and may include nonaromatic rings. Thus, aryl groups include, but are not limited to, benzene, naphthalene, phenyl, biphenyl and the like. The aryl group can be substituted or unsubstituted. The aryl group can be substituted with one or more groups including, but not limited to, substituted or unsubstituted alkyl, cycloalkyl, alkoxy, alkenyl, cycloalkenyl, alkynyl, cycloalkynyl, heterocyclyl, aryl, heteroaryl, aldehyde, amide, amino, carboxylic acid, boronic acid, ester, ether, halide, hydroxy, ketone, azide, nitro, hydroxamate, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “heterocyclyl” as used herein refers to non-aromatic ring compounds containing 3 to 14 ring members, of which one or more is a heteroatom such as, but not limited to, N, O, P and S. In some embodiments, heterocyclyl groups include 3 to 6, 3 to 10, 3 to 12, or 5 to 6 ring members. Heterocyclyl groups encompass partially unsaturated and saturated ring systems, such as, for example, imidazolinyl and imidazolidinyl groups. The phrase “heterocyclyl group” includes fused rings and may include an aromatic group fused to a non-aromatic group, e.g. dihydrobenzofuran. The phrase also includes bridged polycyclic ring systems containing a heteroatom such as, but not limited to, quinuclidinyl. However, the phrase does not include heterocyclyl groups that have other groups, such as alkyl, oxo or halo groups, bonded to one of the ring members. Rather, these are referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, piperidinyl, piperazinyl, morpholinyl, thiomorpholinyl, tetrahydropyranyl, tetrahydrothiopyranyl, quinuclidinyl groups. Representative substituted heterocyclyl groups may be mono-substituted or substituted more than once, such as, but not limited to, piperidinyl or morpholinyl groups, which are 2-, 3-, 4-, 5-, or 6-substituted, or disubstituted with various substituents such as those listed above.

The term “heteroaryl” as used herein refers to aromatic ring compounds containing 5 to 14 ring members, of which, one or more is a heteroatom such as, but not limited to, N, O, P and S. Heteroaryl groups can have one, two or three rings. Heteroaryl groups therefore include, but are not limited to, groups such as pyrrolyl, pyrazolyl, triazolyl, tetrazolyl, oxazolyl, isoxazolyl, thiazolyl, pyridyl, pyridazinyl, pyrimidinyl, pyrazinyl, thiophenyl, benzothiophenyl, furanyl, benzofuranyl, indolyl, azaindolyl (pyrrolopyridyl), indazolyl, benzimidazolyl, imidazopyridyl (azabenzimidazolyl), pyrazolopyridyl, triazolopyridyl, benzotriazolyl, benzoxazolyl, benzothiazolyl, benzothiadiazolyl, imidazopyridyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups. Although the phrase “heteroaryl groups” includes fused ring compounds such as indolyl and 2,3-dihydro indolyl, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

The term “aldehyde” as used herein is represented by the formula —C(O)H. Throughout this specification “C(O)” is a short hand notation for a carbonyl group, i.e., C═O.

The term “amide” (or “amido”) includes C- and N-amide groups, i.e., —C(O)NA¹A², and —NA¹C(O)A² groups, respectively. A¹ and A² are independently hydrogen, or a substituted or unsubstituted alkyl, alkenyl, alkynyl, cycloalkyl, aryl, aralkyl, heterocyclylalkyl or heterocyclyl group as described herein. Amido groups therefore include but are not limited to carbamoyl groups (—C(O)NH₂) and formamide groups (—NHC(O)H).

The terms “amine” or “amino” as used herein are represented by the formula —NA¹A², where A¹ and A² can be, independently, hydrogen or a substituted or unsubstituted alkyl, cycloalkyl, alkenyl, cycloalkenyl, alkynyl, cycloalkynyl, heterocyclyl, aryl, or heteroaryl group as described herein.

The term “boronic acid moiety” as used herein refers to a compound comprising a boronic acid group. The term may be represented by the formula -A¹B(OH)₂, where A¹ can be a substituted or unsubstituted alkyl, cycloalkyl, alkenyl, cycloalkenyl, alkynyl, cycloalkynyl, heterocyclyl, aryl, or heteroaryl group as described herein. Also included within the meaning of this term are ionized compounds, salts, and tetravalent structures.

The term “carboxylic acid” as used herein is represented by the formula —C(O)OH.

The term “ester” as used herein is represented by the formula —OC(O)A¹ or —C(O)OA¹, where A¹ can be a substituted or unsubstituted alkyl, cycloalkyl, alkenyl, cycloalkenyl, alkynyl, cycloalkynyl, heterocyclyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, a substituted or unsubstituted alkyl, cycloalkyl, alkenyl, cycloalkenyl, alkynyl, cycloalkynyl, heterocyclyl, aryl, or heteroaryl group described herein and “a” is an integer from 1 to 500. “Polyester” is also the term used to describe a group that is produced by the reaction between a compound having at least two carboxylic acid groups with a compound having at least two hydroxyl groups.

The term “ether” as used herein is represented by the formula A¹OA², where A¹ and A² can be, independently, a substituted or unsubstituted alkyl, cycloalkyl, alkenyl, cycloalkenyl, alkynyl, cycloalkynyl, heterocyclyl, aryl, or heteroaryl group described herein. The term “polyether” as used herein is represented by the formula -(A¹O-A²O)ₐ—, where A¹ and A² can be, independently, a substituted or unsubstituted alkyl, cycloalkyl, alkenyl, cycloalkenyl, alkynyl, cycloalkynyl, heterocyclyl, aryl, or heteroaryl group described herein and “a” is an integer of from 1 to 500. Examples of polyether groups include polyethylene oxide, polypropylene oxide, and polybutylene oxide.

The term “halide” as used herein refers to the halogens fluorine, chlorine, bromine, and iodine.

The term “hydroxyl” (or “hydroxy”) as used herein is represented by the formula —OH.

The terms “hydroxamate” and “hydroxamic acid moiety” as used herein are represented by the formula -A¹C(O)NHOA²-, where A¹ can be a substituted or unsubstituted alkyl, cycloalkyl, alkenyl, cycloalkenyl, alkynyl, cycloalkynyl, heterocyclyl, aryl, or heteroaryl group as described herein, and A² can be a hydrogen or an alkyl group described herein.

The term “ketone” as used herein is represented by the formula A¹C(O)A², where A¹ and A² can be, independently, a substituted or unsubstituted alkyl, cycloalkyl, alkenyl, cycloalkenyl, alkynyl, cycloalkynyl, heterocyclyl, aryl, or heteroaryl group as described herein.

The term “azide” as used herein is represented by the formula —N₃.

The term “nitro” as used herein is represented by the formula —NO₂.

The term “nitrile” as used herein is represented by the formula —CN.

The term “silyl” as used herein is represented by the formula —SiA¹A²A³, where A¹, A², and A³ can be, independently, hydrogen or a substituted or unsubstituted alkyl, cycloalkyl, alkoxy, alkenyl, cycloalkenyl, alkynyl, cycloalkynyl, heterocyclyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or a substituted or unsubstituted alkyl, cycloalkyl, alkenyl, cycloalkenyl, alkynyl, cycloalkynyl, heterocyclyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or a substituted or unsubstituted alkyl, cycloalkyl, alkenyl, cycloalkenyl, alkynyl, cycloalkynyl, heterocyclyl, aryl, or heteroaryl group as described herein. The term “sulfone” as used herein is represented by the formula -A¹S(O)₂A², where A¹ and A² can be, independently, a substituted or unsubstituted alkyl, cycloalkyl, alkenyl, cycloalkenyl, alkynyl, cycloalkynyl, heterocyclyl, aryl, or heteroaryl group as described herein. The term “sulfoxide” as used herein is represented by the formula -A¹S(O)A², where A¹ and A² can be, independently, a substituted or unsubstituted alkyl, cycloalkyl, alkenyl, cycloalkenyl, alkynyl, cycloalkynyl, heterocyclyl, aryl, or heteroaryl group as described herein.

The term “thiol” as used herein is represented by the formula —SH.

Those of skill in the art will appreciate that compounds of the invention may exhibit the phenomena of tautomerism, conformational isomerism, geometric isomerism and/or optical isomerism. As formula drawings within the specification and claims can represent only one of the possible tautomeric, conformational isomeric, optical isomeric or geometric isomeric forms, it should be understood that the invention encompasses any tautomeric, conformational isomeric, optical isomeric and/or geometric isomeric forms of the compounds having one or more of the utilities described herein, as well as mixtures of these various different forms.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

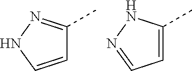

As readily understood by one skilled in the art, a wide variety of functional groups and other structures may exhibit tautomerism, and all tautomers of compounds as described herein are within the scope of the present invention.

Stereoisomers of compounds (also known as optical isomers) include all chiral, diastereomeric, and racemic forms of a structure, unless the specific stereochemistry is expressly indicated. Thus, compounds used in the present invention include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these are all within the scope of the invention.

Reference will now be made in detail to specific aspects of the disclosed methods and materials, compounds, compositions, articles and devices used in the methods, examples of which are illustrated in the accompanying Examples and Figures.

In accordance with one aspect, the present invention provides methods for inhibiting viral or bacterial activity using polymers functionalized with boronic acid moieties. Also provided are methods for treating infections using such functionalized polymers. While not wishing to be limited by theory, it is believed that the functionalized polymers disclosed herein are capable of covalently binding to glycoproteins on the surface of the viruses and bacteria. Specifically, it is believed that the boronic acid moieties on the polymer react with hydroxyl groups of the sugar residues of such glycoproteins to form stable boron-oxygen bonds as shown schematically in Scheme 1. Scheme 1 depicts a substituted phenylboronic acid moiety, although the present invention encompasses unsubstituted phenylboronic acid moieities and other boronic acid moieties.

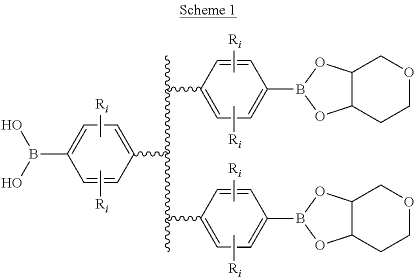

Polymers having multiple boronic acid moieties will bind to the virus or bacterium at multiple sites as illustrated schematically in Scheme 1 and inhibit pathogen activity by, e.g., blocking the ability of the virus to bind to host cell receptors and therefore preventing viral entry and infection. Furthermore this can function with bacteria by binding the surface of the bacteria and inhibiting its ability to interact with cell surfaces and colonize the epithelia surfaces. Due to the multivalent, specific and covalent interactions of these polymers with viruses and bacteria, the methods of the present invention are expected to be more effective at inhibiting activity than compounds that interact only noncovalently with bacteria and viruses.

In one embodiment of the invention, the method for inhibiting the activity of a virus or a bacterium includes contacting a virus or bacterium with a polymer functionalized with one or more boronic acid moieties

The polymers used in the methods of the present invention are functionalized with one or more boronic acid moieties as defined herein. The particular boronic acid moieties used to functionalize the polymers will depend upon the polymer, use, preference and the like. Based on the present disclosure, it is within the ordinary skill in the art to select suitable boronic acid moieties for the use at hand.

Boronic acid moieties are typically derived synthetically from primary sources of boron, such as boric acid. Dehydration of boric acid with alcohols gives rises to borate esters, which are precursors of boronic acid moieties. The secondary oxidation of boranes is also used to prepare boronic acid moieties. Boronic acid moieties can be desirable for the disclosed compositions and methods because of their low toxicity. They also degrade to environmentally friendly boric acid. A discussion of the various methods of preparation and properties of many boronic acid moieties can be found in “Boronic Acids.” Dennis Hall, Ed., Wiley-VCH Verlag, 2005, which is incorporated by reference herein for its teachings of boronic acid derivatives, their preparation, and reactions that involve boronic acid moieties.

In some embodiments of the methods disclosed herein, the one or more boronic acid moieties comprise an alkylboronic acid, where a substituted or unsubstituted, branched or unbranched, alkyl group is substituted with one or more —B(OH)₂ substituents. Thus, some or all of the boronic acid moieties may be an alkylboronic acid. In some embodiments, the alkylboronic acid can have Formula I.

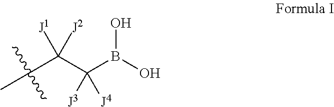

In Formula I, the connection of the alkylboronic acid to the polymer is shown generically by the symbol:

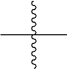

Furthermore, J¹⁻⁴ are independently selected from the group consisting of hydrogen, alkyl, cycloalkyl, alkoxy, alkenyl, cycloalkenyl, alkynyl, cycloalkynyl, heterocyclyl, aryl, heteroaryl, aldehyde, amide, amino, carboxylic acid, ester, ether, halide, hydroxy, azide, nitro, silyl, sulfo-oxo, and thiol substituents. In particular examples of alkylboronic acids, substituents J¹ and J² can both be hydrogen and one of substituents J³ and J⁴ can be hydrogen and the other can be a hydroxy, an alkoxy (e.g., methoxy, ethoxy), a nitro, an amino, or a halide substituent. In other examples, substituents J³ and J⁴ can both be hydrogen and one of substituents J¹ and J² can be hydrogen and the other can be a hydroxy, an alkoxy (e.g., methoxy, ethoxy), a nitro, an amino, or a halide substituent. In a further example, the alkylboronic acid can be a cyclic alkyl (e.g., cyclohexyl) substituted with one or more —B(OH)₂ substituents.

In another embodiment, the one or more boronic acid moieties comprise an arylboronic acid. Thus, some or all of the boronic acid moieties may be an aryl boronic acid. An arylboronic acid contains an aryl group, including heteroaryl groups, as disclosed herein, substituted with one or more —B(OH)₂ substituents. In a specific example, the arylboronic acid can be a phenylboronic acid as shown in Formula II.

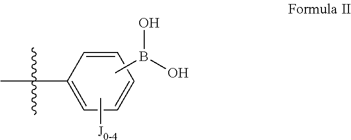

In Formula II, the connection of the arylboronic acid to the polymer is shown generically by the symbol:

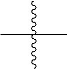

Furthermore, 0 to 4 J substituents are present on the aryl ring and each J is independently selected from the group consisting of substituted or unsubstituted alkyl, cycloalkyl, alkoxy, alkenyl, cycloalkenyl, alkynyl, cycloalkynyl, heterocyclyl, aryl, heteroaryl, aldehyde, amide, amino, carboxylic acid, ester, ether, halide, hydroxy, azide, nitro, silyl, sulfo-oxo, and thiol, trifluoromethyl, benzylamino, benzylalkylamino, benzyloxy and phenylmethylhydroxy. In particular examples of arylboronic acids generally and phenylboronic acids specifically, each substituent J can independently be an ortho hydroxy, ortho hydroxyalkyl, alkoxy (e.g., methoxy, ethoxy), nitro, amino, halide, trifluoromethyl, benzylamino, benzylalkylamino benzyloxy or phenylmethylhydroxy group.

The boronic acid moieties can be attached to the polymers of the present invention directly or by any suitable spacer moiety. A spacer moiety is any compound that provides a link between any of the disclosed polymers and any of the disclosed boronic acid moieties. Examples of spacer moieties include, but are not limited to, alkyl, polyethers, esters, diesters, amides, diamides, and the like. The spacer moieties can be about 1 to about 50 atoms in length (e.g., from 1 to about 25, from about 2 to about 18, from about 4 to about 12, from about 6 to about 10 atoms in length). In some embodiments, the spacer moiety is an amide such as —C(O)NH(CH₂)ₚ or a diamide such as —C(O)NH(CH₂)ₚNHC(O)—, where p is from 1 to 10 (e.g., 3).

The functionalized polymers of the present invention may comprise a wide variety of polymers. The polymers can have a molecular weight of from about 2,000 Da to about 2,000,000 Da. In another aspect, the molecular weight of the polymer can be about 5,000; 10,000; 20,000; 30,000; 40,000; 50,000; 75,000; 100,000; 200,000; 250,000; 300,000; 350,000; 400,000; 450,000; 500,000; 550,000; 600,000; 650,000; 700,000; 750,000; 800,000; 850,000; 900,000; 950,000; 1,000,000; 1,500,000; or 2,000,000 Da, where any stated values can form a lower and/or upper endpoint of a molecular weight range as appropriate.

All or a portion of a polymer suitable for use herein can be hydrophilic or hydrophobic. By “hydrophilic” is meant that the polymer is soluble at or greater than about 1 mg/L of water. By “hydrophobic” is meant that the polymer is soluble at less than about 1 mg/L of water. For example, a hydrophilic polymer can be soluble at about 5 mg/L, 10 mg/L, 50 mg/L, 100 mg/L, 500 mg/L, or greater than 1 g/L. In another example, a hydrophobic polymer can be soluble at about less than about 1 g/L, less than about 0.5 g/L, less than about 0.1 g/L, less than about 0.05 g/L, or less than about 0.01 g/L, or insoluble in water.

A hydrophilic polymer can comprise a homopolymer or a copolymer (e.g., a block, graft, or graft comb copolymer) where one or more of the polymer blocks comprise a hydrophilic segment. In another example, a hydrophobic polymer can comprise a homopolymer or a copolymer (e.g., a block, graft, or graft comb copolymer) where one or more of the polymer blocks comprise a hydrophobic segment. Suitable hydrophilic and hydrophobic polymers and monomers can be obtained from commercial sources or can be prepared by methods known in the art.

Many suitable hydrophilic polymers can form hydrogels. Suitable hydrophilic polymers can include any number of polymers based on diol- or glycol-containing linkages, for example, polymers comprising polyethylene glycol (PEG), also known as polyethylene oxide (PEO), and polypropylene oxide (PPO). Other suitable examples include polymers comprising multiple segments or blocks of PEG alternating with blocks of polyester. For example, POLYACTIVE™ is a copolymer that has large blocks of PEG alternating with blocks of poly(butylene terephthalate). Still other suitable examples include hydrophilic-substituted poly(meth)acrylates, polyacrylates, poly(vinyl alcohol), poly(meth)acrylamides and polyacrylamides, such as poly(hydroxypropyl methacrylamide) and their peglyated copolymers.

Another example of suitable polymers are those that contain a residue of a sulphonamide or sulphonamide derivative. Examples of such polymers include, but are not limited to, copolymers containing aminobenzenesulfonamide (sulfanilamide), and copolymers containing 4-amino-N-[4,6-dimethyl-2-pyrimidinyl]benzene sulfonamide; N-(4-methacrylamido)-N′-(2,6-dimethoxy-4-pyrimydynyl)benzenesulfonamide, 4-amino-N-(4,6-dimethyl-2-pyrimidinyl)benzenesulfonamide; N-(4-methacrylamido)-N′-(4,6-dimethyl-2-pyrimidinyl)benzenesulfonamide; 4-amino-N-(6-methoxy-3-pyridazyl)benzenesulfonamide; N-(4-methacrylamido)-N′-(6-methoxy-3-pyridazyl)benzenesulfonamide; 4-amino-N-(2,6-dimethyl-4-pyrimidinyl)benzenesulfonamide; N-(4-methacrylamido)-N′-(2,6-dimethyl-4-pyrimidinyl)benzenesulfonamide; 4-amino-N-2-pyrinyl benzenesulfonamide; and N-(4-methacrylamido)-N′-2-pyrinyl benzenesulfonamide.

Suitable hydrophobic polymers can include any number of polymers based on olefin, ester, or amide polymerizations. For example, suitable hydrophobic polymers include polyethylene, polypropylene, polybutylene, poly(meth)acrylates, polystyrene, polyamide (e.g., nylon and polycaprolactam), polyacrylonitrile, polyesters, polyurethanes, and the like.

Further examples of hydrophobic polymers are siloxanes, such as decamethylcyclopentasiloxane, octamethylcyclotetrasiloxane, cyclomethicone, dimethicone and mixtures thereof.

In one example, a polymer can comprise a multi-branched polymer (e.g., multi-armed PEG). Multi-branched polymers are polymers that have various polymeric chains (termed “arms” or “branches”) that radiate out from a central core. For example, a suitable hydrophilic polymer can comprise a 2, 3, 4, 5, 6, 7, 8, 9, or 10 armed-PEGs. Such multi-arm polymers are commercially available or can be synthesized by methods known in the art.

Many suitable multi-armed polymers are referred to as dendrimers. The term “dendrimer” means a branched polymer that possesses multiple generations, where each generation creates multiple branch points. “Dendrimers” can include dendrimers having defects in the branching structure, dendrimers having an incomplete degree of branching, crosslinked and uncrosslinked dendrimers, asymmetrically branched dendrimers, star polymers, highly branched polymers, highly branched copolymers and/or block copolymers of highly branched and not highly branched polymers.

Any dendrimer can be used in the disclosed methods. Suitable examples of dendrimers that can be used include, but are not limited to, poly(propyleneimine) (DAB) dendrimers, benzyl ether dendrimers, phenylacetylene dendrimers, carbosilane dendrimers, convergent dendrimers, polyamine, and polyamide dendrimers. Other useful dendrimers include, for example, those described in U.S. Pat. Nos. 4,507,466, 4,558,120, 4,568,737 and 4,587,329, as well as those described in Dendritic Molecules, Concepts, Syntheses, Perspectives. Newkome, et al., VCH Publishers, Inc. New York, N.Y. (1996), which are incorporated by reference herein for at least their teachings of dendrimers

In one example, a suitable polymer comprises a triblock polymer of poly(ethylene oxide)-polypropylene oxide)-poly(ethylene oxide). These polymers are referred to as PLUORONICS™. PLUORONICS™ are commercially available from BASF (Florham Park, N.J.) and have been used in numerous applications as emulsifiers and surfactants in foods, as well as gels and blockers of protein adsorption to hydrophobic surfaces in medical devices. These materials have low acute oral and dermal toxicity, and do not cause irritation to eyes or inflammation of internal tissues in man. The hydrophobic PPO block adsorbs to hydrophobic (e.g., polystyrene) surfaces, while the PEO blocks provide a hydrophilic coating that is protein-repellent. PLUORONICS™ have low toxicity and are approved by the FDA for direct use in medical applications and as food additives. Surface treatments with PLUORONICS™ can also reduce platelet adhesion, protein adsorption, and bacterial adhesion.

In another example, a suitable polymer can comprise a triblock polymer of poly(ethylene oxide)-polypropylene oxide)-poly(ethylene oxide), wherein the polymer has a molecular weight of from 1,000 Da to 100,000 Da. In still another example, a suitable polymer is a triblock polymer of poly(ethylene oxide)-polypropylene oxide)-poly(ethylene oxide), wherein the polymer has a molecular weight having a lower endpoint of 1,000 Da, 2,000 Da, 3,000 Da, 5,000 Da, 10,000 Da, 15,000 Da, 20,000 Da, 30,000 and an upper endpoint of 5,000 Da, 10,000 Da, 15,000 Da, 20,000 Da, 25,000 Da, 30,000 Da, 40,000 Da, 50,000 Da, 60,000 Da, 70,000 Da, 80,000 Da, 90,000 Da, or 100,000 Da, wherein any lower endpoint can be matched with any upper endpoint, wherein the lower endpoint is less than the upper endpoint. In a further example, a suitable polymer can comprise a triblock polymer of poly(ethylene oxide)-polypropylene oxide)-poly(ethylene oxide), wherein the polymer has a molecular weight of from 5,000 Da to 15,000 Da. In yet a further example, the triblock polymer of poly(ethylene oxide)-polypropylene oxide)-poly(ethylene oxide) is PEO103-PPO39-PEO103, PEO132-PPO50-PEO132, or PEO100-PPO65-PEO100. In yet another example, the polymer is PEO103-PPO39-PEO103, PEO132-PPO50-PEO132, or PEO100-PPO 65-PEO100.

Additional polymers can be those based on acrylic acid derivatives, such homopolymers or copolymers of poly(meth)acrylate, polyvinyl alcohol, polyacrylonitrile, polyacrylamides, poly(alkylcyanoacrylates), and the like. Still other examples include polymers based on organic acids such as, but not limited to, polyglucuronic acid, polyaspartic acid, polytartaric acid, polyglutamic acid, polyfumaric acid, polylactide, and polyglycolide, including copolymers thereof. For example, polymers can be made from lactide and/or glycolide monomer units along with a polyether hydrophilic core segment as a single block in the backbone of the polymer. Suitable polymers that are based on esters include, but are not limited to, poly(ortho esters), poly(block-ether esters), poly(ester amides), poly(ester urethanes), polyphosphonate esters, polyphosphoesters, polyanhydrides, and polyphosphazenes, including copolymers thereof.

Still further examples of suitable polymers include, but are not limited to, polyhydroxyalkanoates, poly(propylene fumarate), polyvinylpyrrolidone, polyvinyl polypyrrolidone, polyvinyl-N-methylpyrrolidone, hydroxypropylcellulose, methylcellulose, sodium alginate, gelatin, acid-hydrolytically-degraded gelatin, agarose, carboxymethylcellulose, carboxypolymethylene, poly(hydroxypropyl methacrylate), poly(hydroxyethyl methacrylate), poly(methacryoyl phostidylcholine) and poly(2-hydroxypropyl methacrylamide) and their copolymers.

Some suitable polymers are those that form hydrogels. Examples of hydrogels include, but are not limited to, aminodextran, dextran, DEAE-dextran, chondroitin sulfate, dermatan, heparan, heparin, chitosan, polyethyleneimine, polylysine, dermatan sulfate, heparan sulfate, alginic acid, pectin, carboxymethylcellulose, hyaluronic acid, agarose, carrageenan, starch, polyvinyl alcohol, cellulose, polyacrylic acid, polyacrylamide, polyethylene glycol, or the salt or ester thereof, or a mixture thereof. In one example, the hydrogel can comprise carboxymethyl dextran having a molecular weight of from 5,000 Da to 100,000 Da, 5,000 Da to 90,000 Da; 10,000 Da to 90,000 Da; 20,000 Da to 90,000 Da; 30,000 Da to 90,000 Da; 40,000 Da to 90,000 Da; 50,000 Da to 90,000 Da; or 60,000 Da to 90,000 Da. Still other examples of hydrogels include, but are not limited to, poly(N-isopropyl acrylamide), poly(hydroxy ethylmethacrylate), poly(vinyl alcohol), poly(acrylic acid), polyethylene glycol diacrylate, polyethylene glycol dimethacrylate, and combinations thereof.

In further examples, a suitable polymer can be a polysaccharide. Any polysaccharide known in the art can be used herein. Examples of polysaccharides include starch, cellulose, glycogen or carboxylated polysaccharides such as alginic acid, pectin, carboxymethyl amylose, or carboxymethylcellulose. Further, any of the polyanionic polysaccharides disclosed in U.S. Patent No. 6,521,223, which is incorporated by reference in its entirety, can be used as a suitable polymer or residue thereof. In one example, the polysaccharide can be a glycosaminoglycan (GAG). A GAG is one molecule with many alternating subunits. For example, hyaluronan is (GlcNAc-GlcUA-)ₓ. Other GAGs are sulfated at different sugars. Generically, GAGs are represented by Formula III: A-B-A-B-A-B, where A is an uronic acid and B is an aminosugar that is either O- or N-sulfated, where the A and B units can be heterogeneous with respect to epimeric content or sulfation.

There are many different types of GAGs, having commonly understood structures, which, for example, are within the disclosed compositions, such as chondroitin, chondroitin sulfate, dermatan, dermatan sulfate, heparin, or heparan sulfate. Any GAG known in the art can be used in any of the methods described herein. Glycosaminoglycans can be purchased from Sigma, and many other biochemical suppliers. Alginic acid, pectin, and carboxymethylcellulose are among other carboxylic acid containing polysaccharides useful in the methods described herein.

In one example, the polysaccharide is hyaluronan (HA). HA is a non-sulfated GAG. Hyaluronan is a well known, naturally occurring, water soluble polysaccharide composed of two alternatively linked sugars, D-glucuronic acid and N-acetylglucosamine. The polymer is hydrophilic and highly viscous in aqueous solution at relatively low solute concentrations. It often occurs naturally as the sodium salt, sodium hyaluronate. Other salts such as potassium hyaluronate, magnesium hyaluronate, and calcium hyaluronate, are also suitable. Methods of preparing commercially available hyaluronan and salts thereof are well known. Hyaluronan can be purchased from Seikagaku Company, Clear Solutions Biotech, Inc., Pharmacia Inc., Sigma Inc., and many other suppliers. High molecular weight hyaluronan is often in the range of about 100 to about 10,000 disaccharide units. In another aspect, the lower limit of the molecular weight of the hyaluronan is about 1,000 Da, 2,000 Da, 3,000 Da, 4,000 Da, 5,000 Da, 6,000 Da, 7,000 Da, 8,000 Da, 9,000 Da, 10,000 Da, 20,000 Da, 30,000 Da, 40,000 Da, 50,000 Da, 60,000 Da, 70,000 Da, 80,000 Da, 90,000 Da, or 100,000 Da, and the upper limit is about 200,000 Da, 300,000 Da, 400,000 Da, 500,000 Da, 600,000 Da, 700,000 Da, 800,000 Da, 900,000 Da, 1,000,000 Da, 2,000,000 Da, 4,000,000 Da, 6,000,000 Da, 8,000,000 Da, or 10,000,000 Da, where any of the lower limits can be combined with any of the upper limits.

It is also contemplated that a suitable polymer can have hydrolysable or biochemically cleavable groups incorporated into the polymer network structure. Examples of such hydrogels are described in U.S. Pat. Nos. 5,626,863, 5,844,016, 6,051,248, 6,153,211, 6,201,065, 6,201,072, all of which are incorporated herein by reference in their entireties.

As disclosed above, the polymers of the present invention are functionalized with at least one boronic acid moiety. In some embodiments of the method, the functionalized polymer comprises 2-hydroxypropylmethacrylamide. In other embodiments, the functionalized polymer comprises 2-hydroxypropylmethacrylamide and the one or more boronic acid moieties is phenylboronic acid. The phenylboronic acid may be substituted or unsubstituted. Possible substituents include, but are not limited to, any of the electron withdrawing groups described above. In some embodiments, the substitutents are ortho hydroxymethyl groups. In other embodiments, the substitutents are ortho aminomethyl groups. The 2-hydroxypropylmethacrylamide may be present in a percent molar ratio to phenylboronic acid from about 85:15 to about 99:1. In other embodiments, the ratio is from about 5:95 to about 95:5. In some embodiments, the percent molar ratio of 2-hydroxypropylmethacrylamide to phenylboronic acid is from about 20:80 to about 80:20. In other embodiments, the ratio is from about 25:75 to about 75:25. In still other embodiments, the ratio is from about 35:65 to about 65:35, or from about 45:55 to about 55:45. In yet further embodiments, the ratio is from about 88:12 to about 92:8 and in still other embodiments, the ratio is about 90:10.

The polymers disclosed above may be functionalized with boronic acid moieties in various ways. For example, a monomer containing a particular boronic acid moiety can be polymerized to form a functionalized polymer or a segment of a functionalized polymer. Alternatively, a functional group on a suitable polymer can be converted chemically to a boronic acid. For example, cyclo(ethylene)ester boronates can be hydrolyzed to boronic acid. In another example, the boronic acid can be produced by lithiation of a suitable aryl halide followed by reaction with a protected boron hydride or diboronate.

The polymers described herein can be in the form of a pharmaceutically acceptable salt or ester thereof, provided they possess groups that are capable of being converted to a salt or ester. Pharmaceutically acceptable salts are prepared by treating the free acid with an appropriate amount of a pharmaceutically acceptable base. Representative pharmaceutically acceptable bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like. In other examples, if the polymer possesses a basic group, it can be protonated with an acid such as HCl or H₂SO₄ to produce the cationic salt. The polymer can also be protonated with tartaric acid or acetic acid to produce the tartrate or acetate salt, respectively.

The reaction of the polymer with the acid or base may be conducted in water, alone or in combination with an inert, water-miscible organic solvent, at a temperature of from about 0° C. to about 100° C., such as at room temperature. In certain situations, where applicable, the molar ratio of the disclosed polymers to acid or base is chosen to provide the ratio desired for any particular salts.

Ester derivatives are typically prepared as precursors to the acid form of the compounds and accordingly can serve as prodrugs. Generally, these derivatives will be lower alkyl esters such as methyl, ethyl, and the like.

The polymers disclosed herein may be functionalized with other moieties in addition to boronic acid moieties. For example, the polymers may be functionalized with anionic, cationic, or peglyated functionality. Furthermore, the polymers may be functionalized with biomolecules like bioactive proteins, and therapeutic agents attached to cleavable linkers. Any of the functionalities on the polymer can be used to couple other compounds to the polymer, including, but not limited to, bioactive agents as disclosed below.

The activity of any virus or bacterium which have sugar residues on the surface of the virus or bacterium may be inhibited by exposure to a polymer having boronic acid moieties as described herein. The viral and bacterial activity inhibited includes one or more of subsistence, growth, replication, and infectivity of the organism. In some embodiments, the activity inhibited is the entry of the virus or bacterium into a cell. Likewise, infections by any virus or bacterium which have sugar residues on the surface of the virus or bacterium may be treated or prevented by administering to a subject in need thereof a polymer having boronic acid moieties as described herein. Subjects in need thereof include subjects actually suffering from a viral or bacterial infection, as well as subjects susceptible to such infections. Subjects susceptible to such infections are subjects who have been or will be exposed to any of the viruses or bacteria disclosed herein.

Viruses and bacteria which may be inhibited or treated by methods disclosed herein include those which cause sexually transmitted infections such as human immunodeficiency virus types 1 and 2 (AIDS), human pampalomavirus (HPV), herpes simplex virus (HSV) types 1 and 2, Haemophilus ducreyi (chancroid), chlamydia trachomatis (chlamydia infection or lymphogranuloma venereum (LGV), caused by serotypes L1, L2, and L3) granuloma inguinale or calymmatobacterium granulomatis (donovanosis), neisseria gonorrhoeae (gonorrhea), ureaplasma urealyticum or mycoplasma hominis (non-gonococcal urethritis (NGU)), treponema pallidum (syphilis), and hepatitis B virus (hepatitis B). Hence viruses and bacteria causing such infections include for example retroviruses such as human immunodeficiency virus (HIV-1 and 2). The term “HIV” is meant to encompass all strains of HIV, including, but not limited to, R5 and X4 tropic strains.

In some embodiments, the method further comprises the step of administering one or more bioactive agents to the subject. The bioactive agents are capable of providing a local or systemic biological, physiological, or therapeutic effect in the subject to which it is applied. For example, a bioactive agent can act to control or prevent infection or inflammation, enhance cell growth and tissue regeneration, control tumor growth, act as an analgesic, promote anti-cell attachment, and enhance bone growth, among other functions. Other suitable bioactive agents can include anti-viral agents, hormones, antibodies, or therapeutic proteins. Still other bioactive agents include prodrugs, which are agents that are not biologically active when administered but upon administration to a subject are converted to bioactive agents through metabolism or some other mechanism.

In some embodiments, the bioactive agents can include substances capable of preventing an infection systemically in the subject or locally at the site of infection. For example, antiviral agents against HIV may be used that interfere with one or more steps of the viral life cycle such as entry of HIV into target cells, reverse transcription of the viral genome, integration of the proviral DNA. These include agents which interact with CD4, CXCR4 and CCR5 receptors, or inhibit HIV GP41, HIV NCP7 , HIV GP120, HIV-reverse transcriptase, C-type lectin inhibitors, and HIV-V3 loop inhibitors. Exemplary bioactive agents include but are not limited to BMS-806, mAb 2G12, CD4-IgG2, mAb b12, PRO2000, Dextrin-2-sulphate, cellulose sulphate, polysulphostyrene, caregeenan, cellulose acetate phthalate, cyanovirin, plant lectins, nonoxynol-9, T-20, PSC-Rantes, SCH-C, SCH-D, UK-427, UK-857, AMD-3100, efavirenz, etravirine (TMC-125) rilpivirine (TMC-278), dapiravine, tenofovir and UC-781. Agents that interfere with the integrity of the virus itself may also be used. These viral membrane disruptors such as surfactants, nonoxynol-9, sodium laurel sulphate C31G, cyclodextrins,

Additional bioactive agents that may be co-administered with polymers described herein include anti-inflammatory agents such as, but not limited to, pilocarpine, hydrocortisone, prednisolone, cortisone, diclofenac sodium, indomethacin, 6∝-methyl-prednisolone, corticosterone, dexamethasone, prednisone, and the like; antibacterial agents including, but not limited to, penicillin, cephalosporins, bacitracin, tetracycline, doxycycline, gentamycin, chloroquine, vidarabine, and the like; analgesic agents including, but not limited to, salicylic acid, acetaminophen, ibuprofen, naproxen, piroxicam, flurbiprofen, morphine, and the like; local anesthetics including, but not limited to, cocaine, lidocaine, benzocaine, and the like; immunogens (vaccines) for stimulating antibodies against hepatitis, influenza, measles, rubella, tetanus, polio, rabies, and the like; peptides including, but not limited to, leuprolide acetate (an LH-RH agonist), nafarelin, and the like.

Additionally, a substance or metabolic precursor which is capable of promoting growth and survival of cells and tissues or augmenting the functioning of cells is useful, as for example, a nerve growth promoting substance such as a ganglioside, a nerve growth factor, and the like; a hard or soft tissue growth promoting agent such as fibronectin (FN), human growth hormone (HGH), a colony stimulating factor, bone morphogenic protein, platelet-derived growth factor (PDGF), insulin-derived growth factor (IGF-I, IGF-II), transforming growth factor-α (TGF-α), transforming growth factor-β (TGF-β), epidermal growth factor (EGF), fibroblast growth factor (FGF), interleukin-1 (IL-1), vascular endothelial growth factor (VEGF) and keratinocyte growth factor (KGF), dried bone material, and the like; and antineoplastic agents such as methotrexate, 5-fluorouracil, adriamycin, vinblastine, cisplatin, tumor-specific antibodies conjugated to toxins, tumor necrosis factor, and the like.

Other useful substances include hormones such as progesterone, testosterone, and follicle stimulating hormone (FSH) (birth control, fertility-enhancement), insulin, and the like; antihistamines such as diphenhydramine, and the like; cardiovascular agents such as papaverine, streptokinase and the like; anti-ulcer agents such as isopropamide iodide, and the like; bronchodilators such as metaproternal sulfate, aminophylline, and the like; vasodilators such as theophylline, niacin, minoxidil, and the like; central nervous system agents such as tranquilizer, B-adrenergic blocking agent, dopamine, and the like; antipsychotic agents such as risperidone, narcotic antagonists such as naltrexone, naloxone, buprenorphine; and other like substances. All of these agents are commercially available from suppliers such as Sigma Chemical Co. (Milwaukee, Wis.).

Bioactive agents may also include anti-adhesion compounds. The term “anti-adhesion compound,” as referred to herein, is defined as any compound that prevents cell attachment, cell spreading, cell growth, cell division, cell migration, or cell proliferation. In some examples, compounds that induce apoptosis, arrest the cell cycle, inhibit cell division, and stop cell motility can be used as the anti-adhesion compound. Examples of anti-adhesion compounds include, but are not limited to, anti-cancer drugs, anti-proliferative drugs, PKC inhibitors, ERK or MAPK inhibitors, cdc inhibitors, antimitotics such as colchicine or taxol, DNA intercalators such as adriamycin or camptothecin, or inhibitors of PI3 kinase such as wortmannin or LY294002. In one example, the anti-adhesion compound is a DNA-reactive compound such as mitomycin C. In another example, any of the oligonucleotides disclosed in U.S. Pat. No. 6,551,610, which is incorporated by reference in its entirety, can be used as the anti-adhesion compound. In another example, any of the anti-inflammatory agents described above can be the anti-adhesion compound. Examples of other anti-inflammatory agents include, but are not limited to, methyl prednisone, low dose aspirin, medroxy progesterone acetate, and leuprolide acetate.

Other useful bioactive agents include prohealing compounds. The term “prohealing compound” as defined herein is any compound that promotes cell growth, cell proliferation, cell migration, cell motility, cell adhesion, or cell differentiation. In one example, the prohealing compound includes a protein or synthetic polymer. Proteins useful in the methods described herein include, but are not limited to, an extracellular matrix protein, a chemically-modified extracellular matrix protein, or a partially hydrolyzed derivative of an extracellular matrix protein. The proteins can be naturally occurring or recombinant polypeptides possessing a cell interactive domain. The protein can also be mixtures of proteins, where one or more of the proteins are modified. Specific examples of proteins include, but are not limited to, collagen, elastin, decorin, laminin, or fibronectin.

In another example, the prohealing compound can be any of the supports disclosed in U.S. Pat. No. 6,548,081 B2, which is incorporated by reference in its entirety. In one example, the prohealing compound includes crosslinked alginates, gelatin, collagen, crosslinked collagen, collagen derivatives, such as, succinylated collagen or methylated collagen, cross-linked hyaluronan, chitosan, chitosan derivatives, such as, methylpyrrolidone-chitosan, cellulose and cellulose derivatives such as cellulose acetate or carboxymethyl cellulose, dextran derivatives such carboxymethyl dextran, starch and derivatives of starch such as hydroxyethyl starch, other glycosaminoglycans and their derivatives, other polyanionic polysaccharides or their derivatives, polylactic acid (PLA), polyglycolic acid (PGA), a copolymer of a polylactic acid and a polyglycolic acid (PLGA), lactides, glycolides, and other polyesters, polyoxanones and polyoxalates, copolymer of poly(bis(p-carboxyphenoxy)propane)anhydride (PCPP) and sebacic acid, poly(L-glutamic acid), poly(D-glutamic acid), polyacrylic acid, poly(DL-glutamic acid), poly(L-aspartic acid), poly(D-aspartic acid), poly(DL-aspartic acid), polyethylene glycol, copolymers of the above listed polyamino acids with polyethylene glycol, polypeptides, such as, collagen-like, silk-like, and silk-elastin-like proteins, polycaprolactone, poly(alkylene succinates), poly(hydroxy butyrate) (PHB), poly(butylene diglycolate), nylon-2/nylon-6-copolyamides, polydihydropyrans, polyphosphazenes, poly(ortho ester), poly(cyano acrylates), polyvinylpyrrolidone, polyvinyl alcohol, poly casein, keratin, myosin, and fibrin. In another example, highly crosslinked HA can be the prohealing compound.

In another example, the prohealing compound can be a polysaccharide. In one aspect, the polysaccharide has at least one group, such as a carboxylic acid group or the salt or ester thereof that can react with a boronic acid moiety and/or hydroxamic acid moiety as disclosed herein. In one example, the polysaccharide is a glycosaminoglycan (GAG). Any of the glycosaminoglycans described above can be used in this example. In another example, the prohealing compound is hyaluronan.

The bioactive agent may be administered to the subject before, during or after the administration of the composition containing the functionalized polymer. In other embodiments, the composition containing the functionalized polymer comprises the bioactive agent. In some embodiments, the bioactive agent is noncovalently linked to the functionalized polymer. Noncovalent interactions can include electrostatic or hydrophobic interactions between the bioactive agent and the functionalized polymer. For example, cationic groups (e.g., amino groups) on the functionalized polymer or groups on the functionalized polymer that can be converted to a cationic group can form noncovalent interactions with groups on the bioactive agent that are negatively charged. Conversely, anionic groups (e.g., carboxylic acids or alcohols) on the functionalized polymer or groups on the functionalized polymer that can be converted to an anionic group can form noncovalent interactions with groups on the bioactive agent that are positively charged.

In other embodiments, the bioactive agent is covalently linked to the functionalized polymer. A bioactive agent can be linked to the functionalized polymer through an ether, imidate, thioimidate, ester, amide, thioether, thioester, thioamide, carbamate, disulfide, hydrazide, hydrazone, oxime ether, oxime ester, or and amine linkage. For example, carboxylate-containing chemicals such as the anti-inflammatory agents ibuprofen and hydrocortisone-hemisuccinate can be converted to the corresponding N-hydroxysuccinimide (NHS) active esters and can further react with an OH group of a functionalized polymer. Finally, bioactive agents may also react with boronic acid moieties, hydroxamic acid moieties or other reactive moieties on the functionalized polymers

In some embodiments, the compositions used in the disclosed methods of treatment are in the form of an ointment, lotion, cream, gel, foam, drop, suppository, intravaginal ring, film, spray, liquid or powder. The compositions can be administered to the subject in a number of ways depending on whether local or systemic treatment is desired and on the area to be treated. In some embodiments, the compositions are administered to the vagina or rectum of the subject. Other administration routes include, but are not limited to, oral, buccal, and mucosal.

The compositions used in the disclosed methods of treatment can be formulated in any excipient the subject can tolerate. Examples of such excipients include, but are not limited to, water, saline, Ringer's solution, dextrose solution, Hank's solution, and other aqueous physiologically balanced salt solutions. Nonaqueous vehicles, such as fixed oils, vegetable oils such as olive oil and sesame oil, triglycerides, propylene glycol, polyethylene glycol, and injectable organic esters such as ethyl oleate can also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosol, cresols, formalin, and benzyl alcohol.

The compositions used in the disclosed methods of treatment may be administered topically and can be incorporated into a delivery device. For example, a delivery device may be coated with the compositions disclosed herein. Delivery devices include vaginal devices such as a vaginal tampon, vaginal ring, vaginal strip, vaginal capsule, vaginal tablet, vaginal pessary, vaginal cup, vaginal film, or vaginal sponge.

Examples of subjects that can be treated with the disclosed compositions include birds and mammals such as mice, rats, cows or cattle, horses, sheep, goats, cats, dogs, and primates, including apes, chimpanzees, organatangs, and humans. Dosing of these subjects is dependent on the severity and responsiveness of the infection to be treated, but will normally be one or more does per day, with course of treatment lasting from several days to several months or until one of ordinary skill in the art determines the delivery should cease. Persons of ordinary skill can easily determine optimum dosage, dosing methodologies and repetition rates.

The present invention, thus generally described, will be understood more readily by reference to the following examples, which are provided by way of illustration and are not intended to be limiting of the present invention.

### EXAMPLES

Starting materials are either commercially available as indicated or readily made using procedures well-known to those of skill in the art. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1

### Synthesis of Functionalized Polymers

Monomer Syntheses

Phenylboronic acid-functionalized monomer was synthesized by symmetric anhydride-mediated amidation of N-(3-aminopropyl)methacrylamide hydrochloride (APMA, Polysciences, Inc., Warrington, Pa.) with 4-carboxyphenylboronic acid (PBA, Frontier Scientific, Inc., Logan, Utah). This is shown below in Scheme 2:

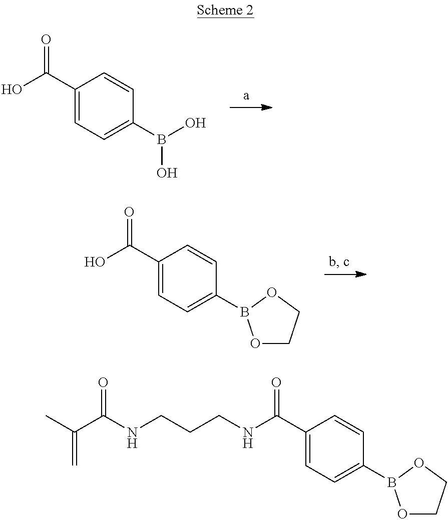

Briefly, PBA was boronate acid-protected using excess (10 eq.) ethylene glycol in dry 1,4-dioxane with molecular sieves present and refluxed for 3 hours at 110° C. (step a). The mixture was then filtered through Celite, concentrated in vacuo, and purified by flash chromatography (96:3:1 CHCl₃:MeOH:AcOH). Pure product (70-85% yield) was confirmed by ¹H NMR. 2.2 eq. of protected PBA was then reacted at room temperature under nitrogen (gas) with 1.1 eq diisopropylcarbodiimide (DIC) in dry 5:2 CH₂Cl₂:DMF for 2 hours (step b) before adding by syringe a mixture of 1 eq. N-(3-aminopropyl)methylacrylamide (APMA), 2 eq. diisopropylethylamine (DIPEA) in minimal dry dimethylformamide (DMF) (step c). The reaction was stirred overnight before concentrating, redissolving in CH₂Cl₂, filtering off precipitated urea side products, and final purification by flash chromatography (95:5 CHCl₃:MeOH). Pure product (73-74% yield) was confirmed by ¹H NMR (CDCl₃): δ 1.99 (s, 3H), 3.42 (m, 2H), 3.50 (m, 2H), 4.385 (s,4H), 5.35 (s,1H), 5.785 (s,1H), 7.84 (m, 4H).

Non-functional vinyl monomer, 2-hydroxypropylmethacrylamide (HPMA), was synthesized by stirring a mixture 1 eq. of 1-amino-2-propanol and 1.5 eq. potassium carbonate in THF at minus 4° C., then adding 1 eq. of methacryloyl chloride dropwise to the chilled mixture, maintaining a reaction temperature below 2° C. After 30 minutes post-addition, the reaction mixture was filtered over Whatman paper, concentrated, redissolved in chloroform and filtered through a silica plug (initially collecting 100% chloroform fractions, followed by 1:9 isopropanol:chloroform fractions until all UV-quenching product was isolated). Following concentration, product was recrystallized from ethyl acetate. Pure product (44% yield) was confirmed by TLC (90:8:2 CHCl₃:MeOH:Acetic acid, R_{f}=0.333) and ¹H NMR (CDCl₃): δ 1.182 (d, 3H), 1.952 (s, 3H), 3.178 (m, 1H), 3.503 (m, 1H), 3.950 (m, 1H), 5.336 (s, 1H), 5.710 (s, 1H), 6.257 (broad s, 1H).

Polymer Syntheses

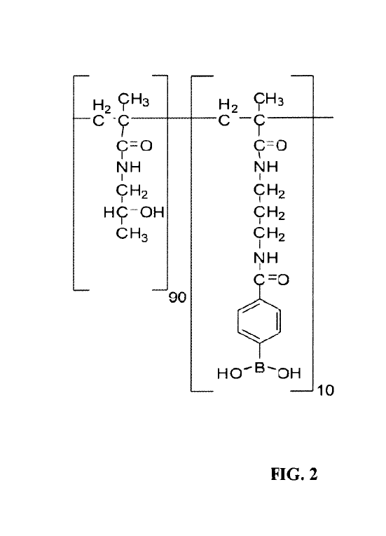

Phenylboronic acid functionalized polymers were synthesized by free radical polymerization of either distilled acrylic acid (AA) or 2-hydroxypropylmethacrylamide (HPMA) and PBA-vinyl (boronic acid protected) monomers from Example 1. Polymerizations of varying degrees of functionalization (5-10 mol % functional monomer) were performed in 75 wt % DMF at 65° C. for 24 hours using 0.6 mol % azo-initiator (AIBN; azobisisobutyronitrile). Some of the polymers are shown below in Table 1 and the structure of one of the polymers is shown in FIG. 2.

TABLE 1

| | Theoretical Molar Ratio | | | |
|---|---|---|---|---|
| | (Actual Molar Ratio*) (mol %) | | | Mw/Mn |
| Polymer | HPMA | AA | PBA vinyl | (kD)** |
| p(HPMA₉₀-PBA₁₀) | 90 (92.6) | — | 10 (7.4) | 451/206 |
| p(AA₉₀-PBA₁₀) | — | 90 (91.5) | 10 (8.5) | 317/254 |

HPMA: 2-hydroxypropylmethacrylamide; AA: acrylic acid; PBA vinyl: N-[3-(2-methyl-acryloylamino)-propyl]-4-amidophenylboronic acid, pinacol ester.
*Actual molar ratio was determined by ¹H NMR in DMSO-d6 (Mercury 400 MHz spectrometer, Varian).
**Mw and Mn were determined by GPC equipped with an aqueous column (PLaquagel-OH mixed, Polymer Labs) or an organic column (PLgel mixed-B, Polymer Labs), a multit-angle light scattering (BI-MwA, Brookhaven Instruments) and differential refractive index detectors (BI-DNDC, Brookhaven Instruments) and are represented as means of at least duplicate experiments (n = 2-6) (GPC 1100, Agilent Technologies). GPC eluents used were either DDI water or HPLC-grade DMF at a flow rate of 0.75 mL/min at 30° C. Polymer samples were injected at a concentration of 0.5 mg/mL.

The boronic acid moieties on the PBA functionalized polymers were deprotected by acidifying the mixtures to pH<4 with 1 M HCl. Polymers were precipitated at least twice in acetone. Finally, polymers were dissolved in DDI water, filtered over 0.45 μm membranes and freeze-dried for at least 72 hours. Polymers (54-76% yield) were characterized by ¹H NMR and GPC.

### Example 2

### Surface Plasmon Resonance Screening of Phenyl Boronic Acid Ligands with HIV gp120

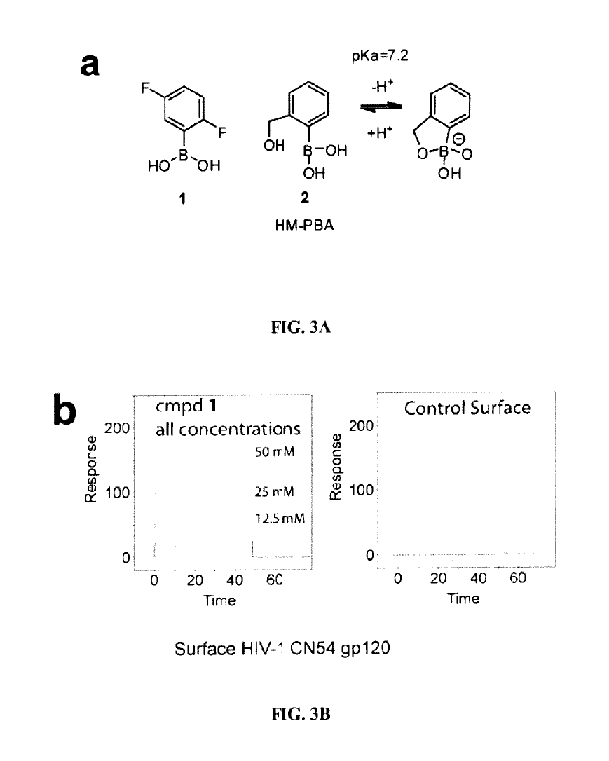

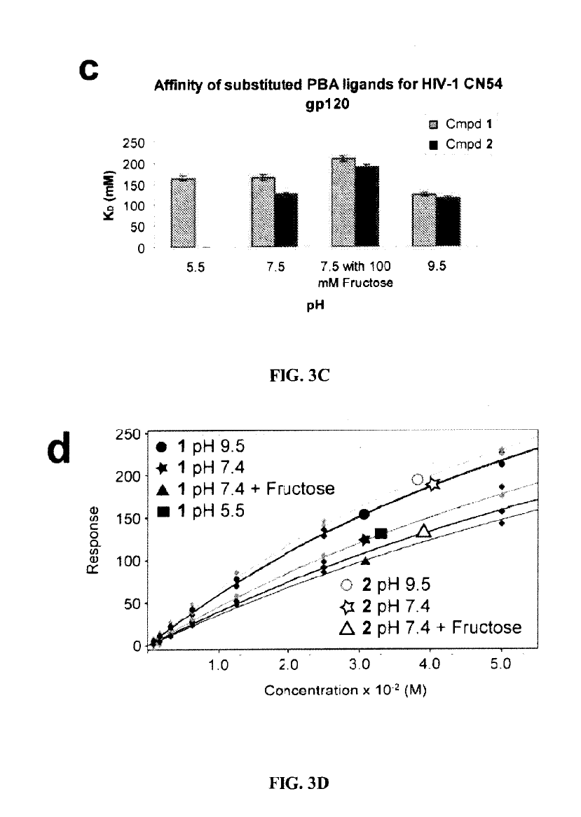

Two commercially available functionalized phenylboronic acids (Frontier Scientific, Logan Utah) were screened to compare the effect of electron withdrawing substituents (2,5-difluorophenylboronic acid, 1, FIG. 3a), versus a ortho-hydroxymethyl substituent (2-hydroxymethylphenylboronic acid, 2, FIG. 3a) prepared at 50 mM concentrations in 25 mM buffers at the appropriate pH (pH 5.5 acetate buffer, pH 7.5 phosphate buffer, and pH 9.5 carbonate buffer). HIV-1 CN54 gp120 with a molecular weight of 130 kDa was obtained from the NIH AIDS Research and Reference Reagent Program (Division of AIDS, NIH). CN54 gp120 was attached to a CM4 sensor chip using standard amine coupling chemistry with a density of 10,000 RU. Briefly, using HBS-P as the running buffer, the carboxymethyl dextran surface of a surface Plasmon resonance (SPR) chip (CM4 chip, Biacore) was activated with an injection of a 1:1 ratio of 0.4M 1-ethyl-3-(3-dimethylaminopropyl)carbodiimide hydrochloride (EDC)/0.1M N-hydroxysuccinimide (NHS). CN54 gp120 in 10 mM sodium acetate (pH 5.5) was then coupled to the surface. The remaining activated groups were then blocked by an injection of 1M ethanolamine (pH 8.5). All coupling and affinity analysis were performed at 25° C. using the Biacore 3000, which has improved signal to noise ratio for samples that exhibit large bulk index of refraction changes. Affinity for the gp120 functionalized surface was determined by comparing raw data to three reference cells: two of the unactivated CM4 surfaces, and an activated CM4 surface capped completely by ethanolamine. The small molecule ligands were injected at the highest concentration prepared, followed by a two-fold dilution series in the appropriate buffer to produce a concentration series. The association and dissociation phases were monitored for 1-3 min and 3-10 min respectively. Data was fitted to a 1:1 interaction model and the apparent affinity, K_{D}, was determined using Scrubber II software. All conditions were repeated at least twice.

Analysis of binding demonstrates that 2 had slightly higher affinity compared to the more acidic 1 as expected based on the ability of the ortho-hydroxymethyl to stabilize the tetrahedral conformation. Compound 1 had similar affinity at pH 5.5 and 7.5, showing an ability to bind to HIV even as the vaginal pH reacidifies.

Binding affinities in the presence of the seminal component fructose were also analyzed. These samples were prepared at 50 mM concentrations in 25 mM pH 7.5 phosphate buffer containing 100 mM fructose. This buffer was used for dilutions and as the running buffer. The K_{D} did decrease, but not significantly, suggesting that fructose in semen may not be a significant competitor for binding (FIG. 3c, d). The presence of glucose, for which most PBAs have a decreased affinity compared to fructose, should exhibit even less of an impact. These results illustrate the ability to assess the affinity of small molecule PBA ligands for a gp120 surface under varying conditions, including in the presence of fructose. This data further supports the importance of the ortho-hydroxymethyl moiety in binding of the PBA to nonreducing pyranose sugars.

### Example 3

### Synthesis of 2-hydroxypropylmethacrylamide Functionalized with 2-hydroxylmethylphenylboronic Acid

5-methyacrylamido-2-hydroxymethylphenylboronic Acid Dehydrate (MAAm-2-HMPBA, 3 in Scheme 3).

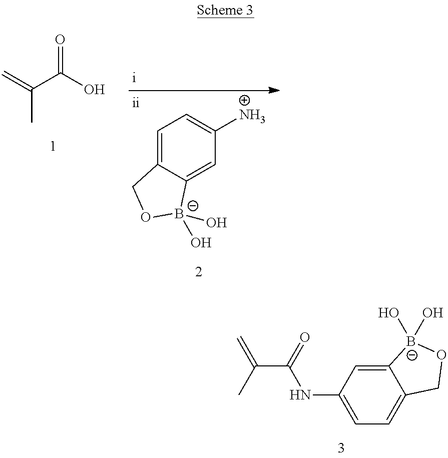

Scheme 3 shows the synthetic conditions for the synthesis of 5-methyacrylamido-2-hydroxymethylphenylboronic acid dehydrate. First the O-Benzotriazole-N,N,N′,N′-tetramethyl-uronium-hexafluoro-phosphate (HBTU) activation of methacrylic acid (MAA-HBTU, i, Scheme 3) was synthesized as follows. Methacrylic acid (1, 0.5256 mL, 6.20 mmol) was dissolved in 25 mL THF Å. HBTU (2.9456 g, 7.78 mmol) and DIPEA (1.127 mL, 6.47 mmol) was added to the solution. Reaction was flushed with N₂ (g) and ran for 4 hrs. TLC showed reaction had gone to completion (MAA, R_{f}=0.7; MAA-HBTU, R_{f}=0.5; HBTU, R_{f}=0; 2:1 Hexane:Ethylacetate 1% Acetic acid). Reaction material was moved forward without any additional work up. In a second round bottom flask, 5-amino-2-hydroxymethylphenylboronic acid HCl dehydrate (2, 1.00 g, 5.39 mmol) was dissolved in THF Å (15 mL). DIPEA (1.88 mL, 10.79 mmol) was added and the solution flushed with N-₂(g) while being chilled in an ice bath. To this the chilled solution, i was added dropwise via syringe while vigorously stirring. Reaction was allowed to warm to ambient temperature and reacted overnight (ii, Scheme 3). Reaction was condensed to a brown oil and redissolved in 4 mL CHCl₃, 200 μL acetic acid and loaded onto a silica column slurry packed in 95:5 CHCl₃:MeOH 1% Acetic acid, with an initial 20 mL CHCl₃ eluted through (dry silica: 150 g, Column dimensions: inner diameter 4.5 cm, height, 23 cm). An additional 20 mL CHCl₃ was eluted through after loading, followed by elution in 95:5 CHCl₃:MeOH 1% acetic acid. Five milliliter fractions were collected. Compound began eluting in fraction 17. Fractions 17-30 were collected and stripped to yield 3 as a yellow oil (1.484 g, 98% yield). ¹H NMR (400 MHz, acetone-d6) δ 9.11 (s, 1H), 8.09 (s, 1H), 7.77 (d, 1H), 7.30 (d, 1H), 5.82 (s, 1H), 5.44 (s, 1H), 4.96 (s, 1H), 1.95 (s, 3H).

2-Hydroxypropylmethacrylamide (HPMAm, 4 in Scheme 4) was synthesized as described above.

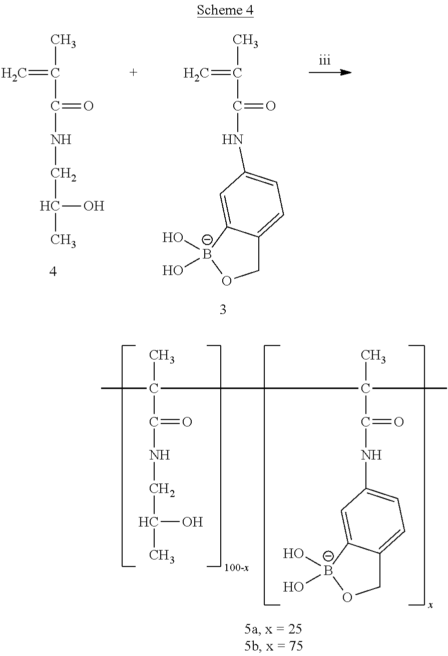

Polymer synthesis (Scheme 4). Polymers were synthesized by free radical polymerizations using approximately 75:25 or 25:75 mole ratios of HPMAm (4) with MAAm-2-HMPBA (3). Polymerizations were performed using 0.5 M solution of monomer with 2,2′-azobisisobutyronitrile (AIBN, 5 mol %) in DMF 4Å at 65° C. for 24 hours under nitrogen atmosphere. During polymerization, 5b, precipitated out of solution and MeOH (200 μL) was added to completely dissolve polymer in order to precipitate. Both polymers solutions were titurated into 40 mL ether, centrifuged at 500 rpm for 5-15 min and supernatant decanted. Polymers were placed under high vacuum overnight to remove residual DMF. Polymers were further purified by dissolving in 100 mM PBS buffer (50.2 mM NaCl, iso-osmolar) to a concentration of 10 mg/mL and centrifuging through a 3,000 MWCU membrane (Amicon Ultra, 4 mL, 3000 MWCU, Millipore, Bellerica, Mass.). 5b required adjusting the buffer to pH 11 in order for complete dissolution to occur. Samples were centrifuged at 3000 rpm for 45 min, filtrate removed and retentate diluted up to initial volume in buffer. This was repeated 3 times with buffer, followed by three times with DI H₂O. Retentate was transferred to falcon tube and lyophilized. Actual molar feed ratios of polymers were determined in D₂O by ¹H NMR (Mercury 400 MHz spectrometer, Varian). 5a was functionalized with a 27.2 mol % of 2-HMPBA and 5b was functionalized with a 69.8 mol % of 2-HMPBA. Polymer molecular distributions were determined in DMF using GPC (GPC 1100, Agilent Technologies, Santa Clara, Calif.) equipped with an organic column (PLgel mixed-B, Polymer Labs, Amherst, Mass.), a differential refractive index detector (BI-DNDC, Brookhaven Instruments, Holtsville, N.Y.) and a multi-angle light scattering detector (BI-MwA, Brookhaven Instruments, Holstville, N.Y.). For 5a the M_{w}/Mₙ was 31,400/17,200.

### Example 4

### Inhibition of HIV Activity by 2-hydroxypropylmethacrylamide Functionalized with 2-hydroxylmethylphenylboronic Acid

Neutralization assay. The procedure for the neutralization assay was derived from Anthony Geonnetti's, et al. Restriction of HIV-1 transport by placebo gel formulations used in microbicide clinical trials. Submitted to: Antimicrobial and Chemotherapeutic Agents.

Media: All solutions were made in D-MEM (Invitrogen, Carlsbad, Calif.) supplemented with 10% fetal bovine serum (FBS, heat-inactivated) (Hyclone, Logan, Utah), 25 mM HEPES (Gibco/Invitrogen, Carlsbad, Calif.) and 50 μg/ml gentamicin (Sigma, St. Louis, Mo.).

Virus Stocks: A R5-tropic, well-characterized, reference strain of HIV-1 isolated from acute, sexually transmitted infections and grown in peripheral blood mononuclear cells (PBMCs) was used: HIV-1 DU156 (Clade C).

Viral Neutralization/Cytotoxity Assays: Polymers were tested for neutralization activity using a previously established and validated assay for testing neutralizing antibodies. {Montefiori, 2004 #420} Polymers were prepared in pH 7.6 100 mM PBS buffer (50.2 mM NaCl, iso-osmolar) at the following concentrations: 5a at 33.33 mg/mL, and 5b at 3.33 mg/mL (this sample had a pH of 7.9). An initial 5-fold dilution of each sample in media occurred (45 μL sample in 205 μL media). Three fold serial dilutions, ranging from 2.22 mg/mL to 0.003 mg/mL for 5a and 0.222 mg/mL to 0.000455 mg/mL for 5b and buffer as a control (18% to 0.0082%) in growth media, were incubated with 350-650 TCID of virus for 60 minutes. 10,000 TZM-b1 cells (100 μL of 1×10⁵ cells/ml in GM with 37.5 μg/ml DEAE dextran) were then added to each well. These cells were a CXCR4-postive HeLa cell clone that was engineered to express CD4 and CCR5 as well as integrated reporter genes for firefly luciferase and E. coli (3-galactosidase under control of an HIV long-terminal repeat sequence. Reporter gene expression was induced by the viral Tat protein soon after single round infection and the total amount of infectious viral particles was directly proportional to the luminescence of the sample. Care was taken to ensure all RLU values were within the linear range of the TZM-b1 assay.

After 48 hr. incubation at 37° C., 150 μL was removed from each well and 100 μL of Britelite Reagent (PerkinElmer, Waltham, Mass.) was added. After 2 minute room temperature incubation, the wells were mixed by pipette action and 150 μL was transferred to a 96-well black plate and read in a luminometer (Wallac 1420 Victor, PerkinElmer, Waltham, Mass.). Relative luminescence values were compared to negative (cells-only, no virus) and positive (cells with virus) controls. Both polymer solutions and buffer alone were also tested for cytotoxicity using the same assay without any virus. In these cytotoxicity assays, the cellular luminescence from each well was compared to cell controls. A drop in well luminescence suggested a sample mediated reduction in cell number.

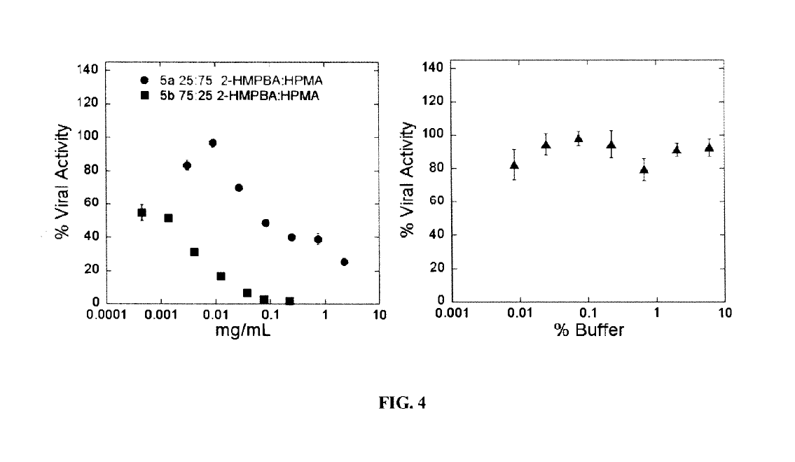

FIG. 4 shows the results from the neutralization assay of polymer solutions 5a and 5b in buffer (Left) compared to buffer alone (Right) under serial dilutions with growth media. Buffer alone exhibited no antiviral activity across the range tested. However 5b showed significant antiviral activity across the range tested, with an EC₅₀ of 1.4 mg/mL. Assuming a M_{w} of around 50,000 Da, that yields an approximate EC₅₀ of 2.7 nM. The lower functionalized 5a provided less antiviral activity with an EC₅₀ of 82 mg/mL (M_{w}=31,400 Da yields an EC₅₀ of 2617 nM.) N=3, Mean±SD.

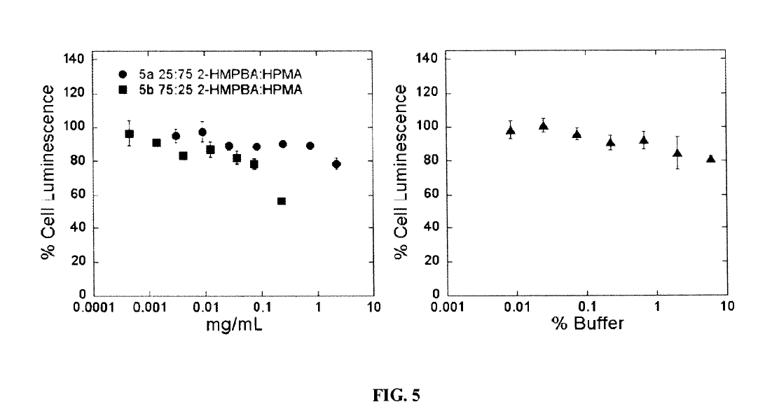

FIG. 5 shows the results from cytotoxicity assay of polymer solutions 5a and 5b in buffer (Left) compared to buffer alone (Right) under serial dilutions with growth media. The luminescent readout was normalized to cells alone. Buffer alone showed slight cytoxicity at the higher percentages. 5a demonstrated a similar profile to buffer alone, whereas, 5b did appear to impact cell viability at the highest concentration. This concentration is well below the EC₅₀ determined from the antiviral assay. This data signifies that 5b is antiviral with minimal cytotoxicity. N=3, Mean±SD.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

What is claimed is:

1. A method comprising contacting a virus or a bacterium with a polymer functionalized with one or more arylboronic acid groups, whereby the activity of the virus or bacterium is inhibited.

2. The method of claim 1, wherein the arylboronic acid group is substituted or unsubstituted phenylboronic acid.

3. The method of claim 1, wherein each arylboronic acid group comprises an ortho hydroxymethyl group.

4. The method of claim 1, wherein the one or more arylboronic acid groups comprises a spacer moiety selected from the group consisting of alkyl, polyether, ester, diester, amide and diamide groups.

5. The method of claim 4, wherein the spacer moiety is —C(O)NH(CH₂)ₚ-, or —C(O)NH(CH₂)ₚNHC(O)—, where p is from 1 to 10.

6. The method of claim 1, wherein the polymer comprises a homopolymer, a copolymer, or a multi-armed polymer.

7. The method of claim 1, wherein the polymer comprises one or more polymers selected from the group consisting of polyethylene oxide and polypropylene oxide.

8. The method of claim 1, wherein the polymer comprises one or more polymers selected from the group consisting of polyethylene, polypropylene, polybutylene, polystyrene, polyamide, polyacrylonitrile, polyester, and polyurethane.

9. The method of claim 1, wherein the polymer comprises one or more polymers selected from the group consisting of polyacrylate, poly(meth)acrylate, poly(hydroxymethyl methacrylate), poly(hydroxyethyl methacrylate), poly(hydroxypropyl methacrylate), poly(alkylcyanoacrylate), polyethylene glycol diacrylate, polyethylene glycol dimethacrylate, polyacrylamide, poly(N-isopropyl acrylamide), poly(meth)acrylamide, and poly(hydroxypropyl methacrylamide).

10. The method of claim 1, wherein the polymer comprises 2-hydroxypropylmethacrylamide.

11. The method of claim 1, wherein the polymer comprises 2-hydroxypropylmethacrylamide and the one or more arylboronic acid groups comprises substituted or unsubstituted phenylboronic acid.

12. The method of claim 11, wherein the one or more arylboronic acid groups comprises 2-hydroxymethyl phenylboronic acid.

13. The method of claim 11, further wherein 2-hydroxypropylmethacrylamide is present in a percent molar ratio to phenylboronic acid of from about 5:95 to about 95:5.

14. The method of claim 1, wherein the virus or bacterium is capable of causing a sexually transmitted infection.

15. The method of claim 1, wherein the virus is selected from the group consisting of HIV, HPV, and HSV.

16. The method of claim 1, wherein the polymer is capable of forming one or more covalent bonds to the virus.

17. A method of treatment comprising administering an effective amount of a composition to a subject in need thereof, the composition comprising a polymer functionalized with one or more arylboronic acid groups.

18. The method of claim 17, wherein the polymer comprises 2-hydroxypropylmethacrylamide and the one or more arylboronic acid groups comprises substituted or unsubstituted phenylboronic acid.

19. The method of claim 17, wherein the subject is suffering from, or is susceptible to a sexually transmitted infection.

20. The method of claim 17, further comprising administering a bioactive agent, wherein the bioactive agent is selected from the group consisting of anti-inflammatory agents, antibacterial agents, analgesic agents, local anesthetics, immunogens, hormones, contraceptive agents, antivirals, antifungals, and antihistamines.
